(19) United States
(12) Patent Application Publication
(10) Pub. No.: US 2024/0415685 A1
(43) Pub. Date: Dec. 19, 2024

(54) SMART DEVICE SUPPORT ASSEMBLY

(71) Applicant: Shawn Shetty, Cambridge, MA (US)

(72) Inventor: Shawn Shetty, Cambridge, MA (US)

(21) Appl. No.: 18/745,953

(22) Filed: Jun. 17, 2024

Related U.S. Application Data
(60) Provisional application No. 63/521,447, filed on Jun. 16, 2023.

(51) Int. Cl. A61F 5/01; A45F 5/00

(52) U.S. Cl. CPC A61F 5/0118; A45F 5/00

(57) ABSTRACT

The present invention provides a wrist brace and a smart device holder supported by the wrist brace, forming a smart device support assembly. The device holder has mechanical functions that allow a user to use a smart device without using their hand. Furthermore, the device support assembly may be used as a medical wrist brace while the smart device holder with mechanical function works synergistically together to provide hands free use and multiple degrees of freedom of the smart device.

### CROSS REFERENCE TO RELATED APPLICATIONS

The present application claims priority to U.S. Provisional Patent Application Ser. No. 63/521,447 filed on Jun. 16, 2023, the contents of which are incorporated herein by reference.

### FIELD OF THE INVENTION

The present invention relates in general to wrist braces with an attached appendage to hold various devices. More particularly, the present invention relates to a wrist brace with an attached appendage to hold a smartphone.

### BACKGROUND OF THE INVENTION

A smart device, such as smartphone, is essential for billions of users worldwide. An adequate hand grip strength is required to access the device and prevent it from dropping it from the holder's hand. However, there are multiple medical conditions that can reduce the hand grip strength such as post-stroke syndrome, multiple sclerosis, Parkinson's Disease, Rheumatoid Arthritis, Osteoarthritis, Carpal Tunnel Syndrome, and other nerve palsies.

Furthermore, wrist and finger injuries can occur due to smart device overuse. Prior to the present invention, individuals did not have a device to help them hold and manage the operation of a smart device by attaching it to an artificial appendage.

Conventional wrist braces do not offer additional mechanical function, and other smart device holders do not offer hands-free use. Thus, conventional wrist braces do not allow people to manage their smart devices without gripping them.

Thus, there is a need for a smart device support assembly that includes a wrist brace with an attached appendage for holding a smart device.

### BRIEF SUMMARY OF THE INVENTION

An objective of the present invention is to provide a smart device support assembly for hands-free use of smart devices. According to various embodiments of the present invention, a smart device support assembly comprises a wrist brace and a smart device holder supported by the wrist brace. The device holder has mechanical functions that allow a user to operate a smart device without using their hand. Furthermore, the device support assembly may be used as a medical wrist brace while the smart device holder with mechanical function works synergistically together to provide hands-free use and multiple degrees of freedom of the smart device.

### BRIEF DESCRIPTION OF THE DRAWINGS

FIG. 1 are isometric views of a smart device holder with different configurations for use as part of a smart device support assembly according to a first embodiment of the present invention, showing an expandable and retractable feature of an appendage attached to a device mount in the smart device holder;

FIG. 2 is an exploded view of the smart device holder of FIG. 1 according to the first embodiment of the present invention, showing various components of the smart device holder;

FIG. 3 is a perspective view of a wrist brace as part of the smart device support assembly of FIG. 1 according to the first embodiment of the present invention, shown in a retracted position;

FIG. 4 is a perspective view of the wrist brace of FIG. 3 according to the first embodiment of the present invention, shown in an expanded position;

FIG. 5 is a front view of the wrist brace of FIG. 3 according to the first embodiment of the present invention, shown in the retracted position;

FIG. 6 is a front view of the wrist brace of FIG. 3 according to the first embodiment of the present invention, shown in the expanded position;

FIG. 7 is an isometric view of a wrist brace and a smart device holder for use as part of a smart device support assembly according to a second embodiment of the present invention;

FIG. 8 are exploded views of inner cuffs of the wrist brace and parts of the appendage of FIG. 7, according to the second embodiment of the present invention;

FIG. 9 is an exploded view of a smart device support assembly according to a third embodiment of the present invention, showing various components of the smartphone support assembly;

FIG. 10 is an isometric view of a smart device support assembly holding a smartphone according to the second or third embodiments of the present invention;

FIG. 11 is an exploded view of a smart device support assembly according to a fourth embodiment of the present invention, showing various components of the smart device support assembly;

FIG. 12 is an isometric back view of the smart device support assembly according to the fourth embodiment of the present invention;

FIG. 13 is an isometric front view of the smart device support assembly according to the fourth embodiment of the present invention;

FIG. 14 is an isometric view of the smart device support assembly according to the fifth embodiment of the present invention;

FIG. 15 is an exploded view of the ring slider assembly applied in the smart device support assembly according to the fifth embodiment of the present invention;

FIG. 16 is an isometric view of an assembled ring slider assembly applied in the smart device support assembly according to the fifth embodiment of the present invention;

FIG. 17 is an isometric view of the smart device support assembly held by a user in the front side of the hand according to the fifth embodiment of the present invention; and

FIG. 18 is an isometric view of the smart device support assembly held by a user in the back side of the hand according to the fifth embodiment of the present invention.

### DETAILED DESCRIPTION OF THE INVENTION

The following detailed description is of the best contemplated modes of carrying out exemplary embodiments of the present invention. The description is not to be taken in a limiting sense but is made merely for the purpose of illustrating the general principles of the present invention.

It should be understood that throughout this patent application and specifically in the written description that follows and in the accompanying drawing figures, various directional and orientational terms such as horizontal, vertical, diagonal, up, down, upward, downward, in, out, inward, outward, forward, backward, sideways, front, rear, back, top, bottom, side, upper, lower, left, right, center, middle, above, below, and other similar directional and orientational terms as they are used herein refer to the smart device support assembly of the various exemplary embodiments of the present invention as that smart device support assembly is oriented when typically being assembled or in use. All these terms are used in this written description and in the drawings for convenience only and as an aid to better understanding the exemplary embodiments of the present invention. These terms are not intended to be limiting in the claims or to imply that the smart device support assembly must be used or positioned in any particular or specific direction or orientation while being assembled or in use to satisfy the limitations of the claims.

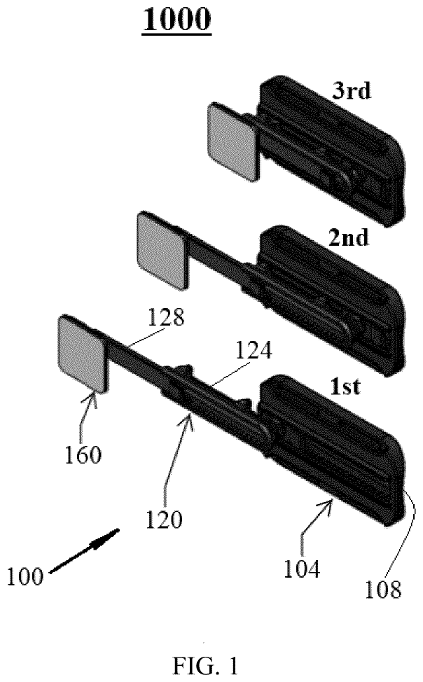

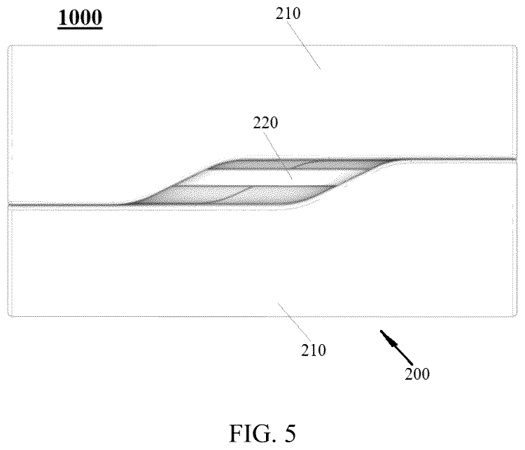

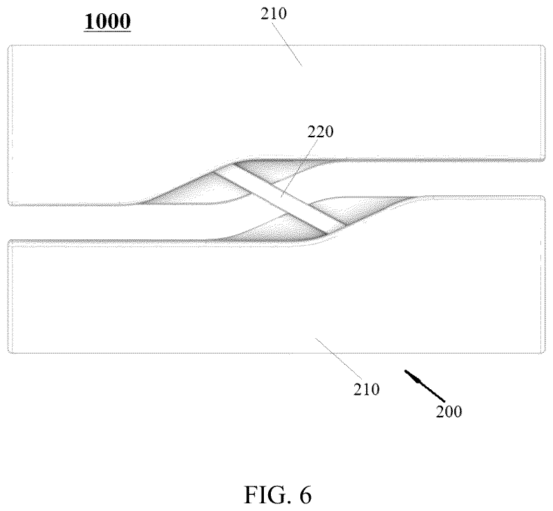

According to various embodiments of the present invention, five different exemplary embodiments of smart device support assemblies of 1000, 2000, 3000, 4000, and 5000 of the present invention have been described in detail herein, accompanied with drawings in FIGS. 1-6, FIGS. 7-8, FIGS. 9-10, FIGS. 11-13, and FIGS. 14-18, respectively.

Referring to FIG. 1, there illustrated are isometric views of a group of device holders 100 with different configurations used as part of a smart device support assembly 1000 according to a first embodiment of the present invention.

Each one of the smart device holders 100 comprises a wrist mount 104, an expandable and retractable appendage 120 with one end attached to the wrist mount 104, and a smart device support member 160 attached to the other end of the appendage 120. The wrist mount 104 includes a glide track 108 to guide sliding movement of the appendage 120.

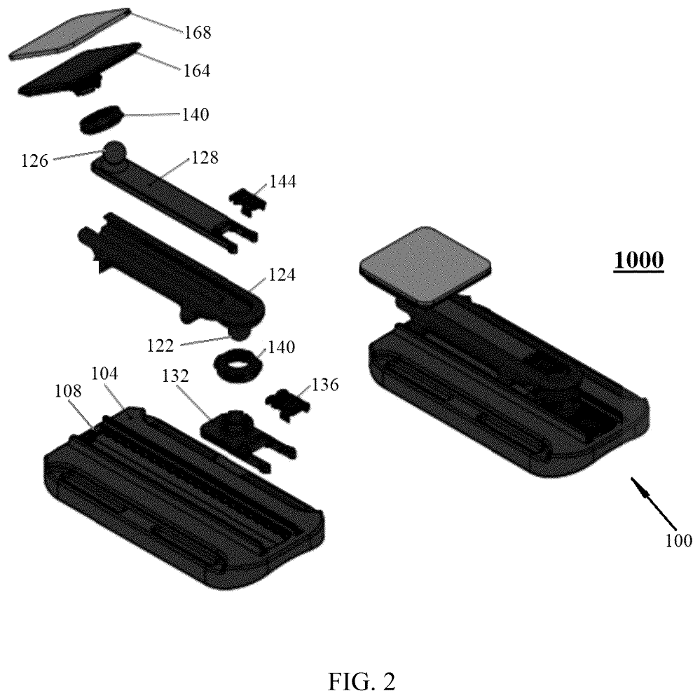

Referring to FIG. 2, there illustrated is an exploded view of the smart device holder 100 according to the first embodiment of the smart device support assembly 1000 of the present invention, showing various components of the smart device holder 100.

As shown in FIG. 2, appendage 120 comprises an outer appendage 124, an inner appendage 128, a slider 132, a first snap fit 136, a tightening nut 140, a second snap fit 144, a first humeral head 122, and a second humeral head 126. The first humeral head 122 is attached to a lower surface of one end of the outer appendage 124, facing downward, and the second humeral head 126 is attached to an upper surface of one end of the inner appendage 128, facing upward.

A bottom section of the slider 132 is fit into the glide track 108 and an upper section of the slider 132 is attached to the humeral head 122 through one of the tightening nuts 140, therefore connecting the slider 132 to the outer appendage 124. Thus, the outer appendage 124 is slidable along the glide track 108. The other end of the outer appendage 124 supports the inner appendage 128 to slide in and out of the outer appendage 124. The first snap fit 136 is fit into the slider 132, functioning to guide and to prevent the slider 132 from sliding off the glide track 108. The second snap fit 144 is fit to one end of the inner appendage 128, functioning to guide and to prevent the inner appendage 128 from sliding off the outer appendage 124. The inner appendage 128 is slidable within the outer appendage 124, making the appendage 120 expandable and retractable.

Referring again to FIG. 2, the smart device support member 160 comprises a device mount 164 and a double-sided tape 168 attached to the device mount 164 for mounting a smart device. The device mount 164 is attached to the second humeral head 126 through another one of the tightening nuts 140, connecting the device mount 164 to the appendage 120.

The device mount 164, which may be made of polycarbonate, is provided with magnets embedded into the device mount 164 and may be approximately 10 cm long and 5 cm wide with ridges. The double-sided tape 168, which is attached to the device mount 164, may be made of a non-permanent adhesive material with sufficient strength to maintain connection between the smart device and the support member 160.

Referring again to FIG. 1, the appendage 120 can be expanded by either pulling the inner appendage 128 outwardly from the outer appendage 124 or sliding the outer appendage 128 forward in the glide track 108. As shown in FIG. 1, the appendage 120 in the 1st smart device holder 100 is in a fully expanded configuration, where the inner appendage 128 is pulled out from the outer appendage 124 and the outer appendage 124 slides forward to the end of the glide track 108; the appendage 120 in the 2nd smart device holder is in a partially expanded configuration, where the inner appendage 120 is pulled out while the outer appendage 124 is retracted to the glide track 108; and the appendage 120 in the 3rd smart device holder 100 is in a fully retracted configuration, where the inner appendage 128 is retracted into the outer appendage 124 and the outer appendage 124 is retracted to the glide track 108.

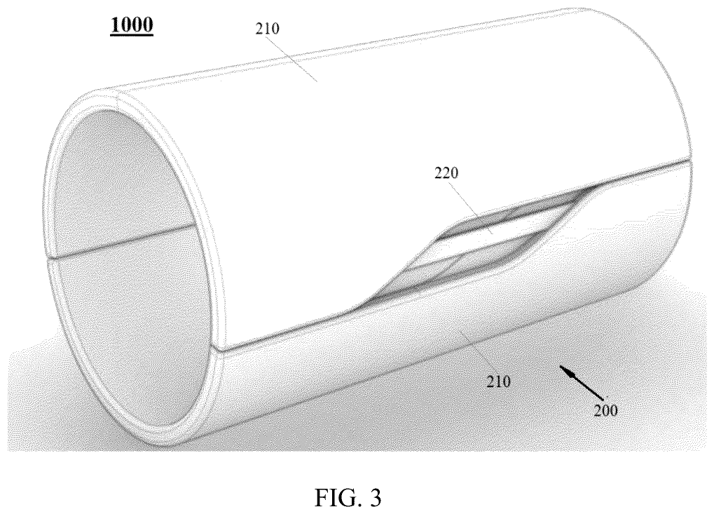

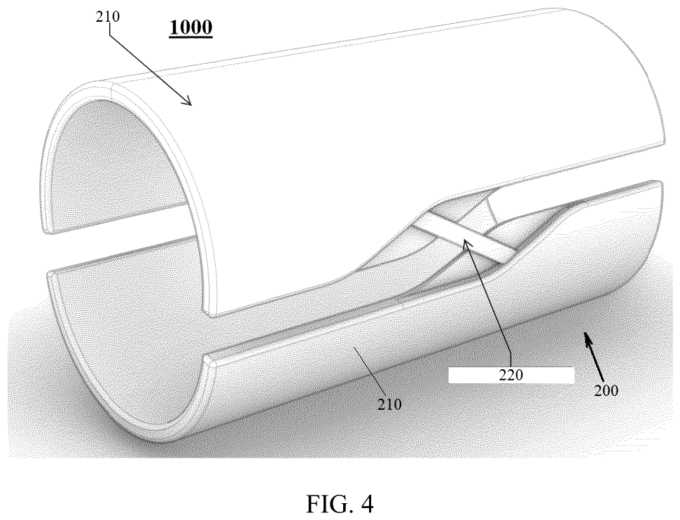

Referring to FIGS. 3-6, there illustrated are perspective views of a wrist brace 200 for the smart device support assembly 1000. The wrist brace 200 comprises two semi-circular cuffs 210 which may be made from polycarbonate. The wrist brace 200 are formed of sufficient length to accommodate an adult wrist from start of wrist to approximately five inches of the forearm. The cuffs 210 are attached to two sliding hinges 220 (which may be metallic), which allow them to be adjustable over the wrist and forearm. As shown, the two semi-circular cuffs 210 are in the retracted and expanded positions in FIGS. 3 and 4, respectively. A clasp may be provided that is adjustable to the size of the forearm. The clasp is attached to one of the two semi-circular cuffs 210. The clasp can be optional, as it would simply decrease the multiple degrees of freedom in which a smart device can be positioned but embodiments of the present invention could function without it.

Referring again to FIG. 2, each end of the appendage 120 is attached to one of the humeral heads 122 or 126, which may be, for example, 25 millimeters (mm) in diameter. The smart device holder 100 is embedded into one of the semi-circular cuffs 210 and is attached to the clasp 230. Further provided is the wrist mount 104 with a metallic base and internal ridges approximately 10 cm long and a socket 25 millimeters (mm) in diameter centered at the bottom of the wrist mount 104. The wrist mount 104 is attached to the slider 132. The slider 132 is attached to the humeral head 122 of the appendage 120.

In use, the device slides over the hand and wrist of a user and fits over the wrist and forearm. The two semi-circular cuffs 210 of the wrist brace 200 slide together and attach over the forearm, so it feels comfortable to the user. The smart device is attached to the polycarbonate device mount 164 and can be moved vertically up and down the mount 164 using fingers of the same hand the device is attached. The appendage 120, when extended, is to be used by the thumb and can then be retracted back along the forearm. To extend the appendage 120, the other hand pulls out the appendage 120. In certain embodiments, the appendage 120 can be removed from the wrist brace 200, in which case the wrist brace 200 would only function as a wrist brace (rather than doubling as a support for a smart device holder). Further, the device could be operated by a computer if electronics were added to the device.

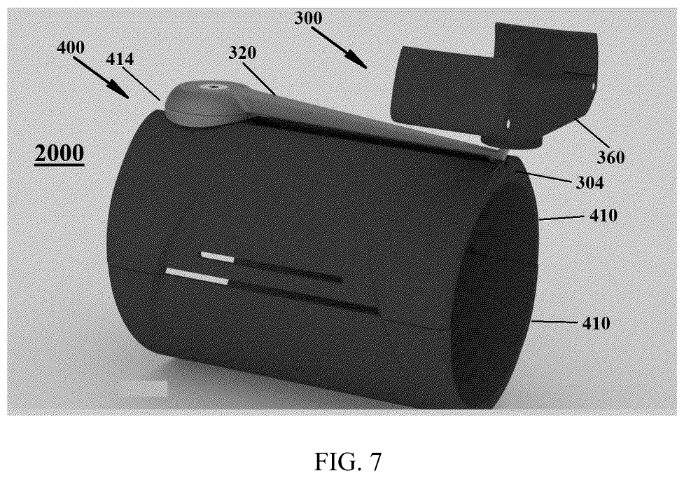

Referring to FIG. 7, there illustrated is a smart device support assembly 2000 according to a second exemplary embodiment of the present invention. Like the smart device support assembly 1000, the smart device support assembly 2000 comprises a device holder 300 and a wrist brace 400. The device holder 300 comprises a wrist mount 304, an appendage 320, and a device mount 360. The device holder 300, which is composed of plastic or a similar material, is held in place by having the appendage 320 inserted into a glide track 414 located on and along the wrist brace 400.

As shown in FIG. 7, one end of the appendage 320 has a rotary joint to allow for a vertical movement along the Y axis from 180 degrees to 45 degrees, where the Y axis is perpendicular to the orientation of the appendage. The appendage 320 that lies inside the glide track 414 can slide horizontally along the X axis as needed by the user for access to different points of the device screen operated by the thumb, where the X axis is parallel to the orientation of the appendage. Magnets lined underneath the glide track 414 allow the appendage 320 to slide back and forth in a segmented fashion.

The other end of the appendage 320 is attached to the device mount 360 placed above a ball joint that allows for rotation of the smart device for easy access to different point along the screen of the smart device.

In FIG. 7, the wrist brace 400 comprises two semi-circular cuffs 410 and a glide track 414 along the wrist brace 400. The wrist brace 400 is made of sufficient length to accommodate an adult wrist from start of wrist to approximately five inches of the forearm. The wrist brace 400 may be made from polycarbonate material and may be custom/personal designed. The semi-circular cuffs 410 may be made of a plastic or similar materials. The two semi-circular cuffs 410 are held together with magnets lined within the material. The two semi-circular cuffs 410 may also be held together through a flexible strap that is also lined within the two semi-circular cuffs. The two cuff pieces 410 when fit together can be clasped using another plastic or similar material piece lined with a magnet to keep the pieces together.

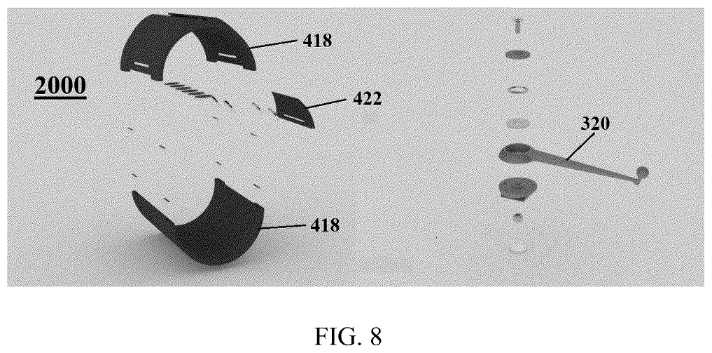

Referring to FIG. 8, there illustrated are two inner cuffs 418 for the smart device support assembly 2000 to function as liners to the wrist brace 400, a clasp 422, and parts of the appendage 320 according to the embodiment of the present invention. The inner cuffs 418 may be lined with comfortable EVA foam. The clasp 422 is provided that is adjustable to the size of the forearm and is attached to one of the two inner cuffs 418.

Referring again to FIG. 8, the parts of the appendage 320 are designed as such that attachment of the appendage 320 to the wrist mount 304 is adjustable so that the smart device can be manually adjusted for different degrees of freedom and provide an enhanced use experience of the smart device.

Both the device holder 300 and the wrist brace 400 of the smart device support assembly 2000 can be changed in terms of function, mechanism, and design. The primary function of both is that 1) the wrist brace 400 provides muscular support and 2) the device holder 300 is an infallible replacement for the hand that can be moved in different degrees of freedom.

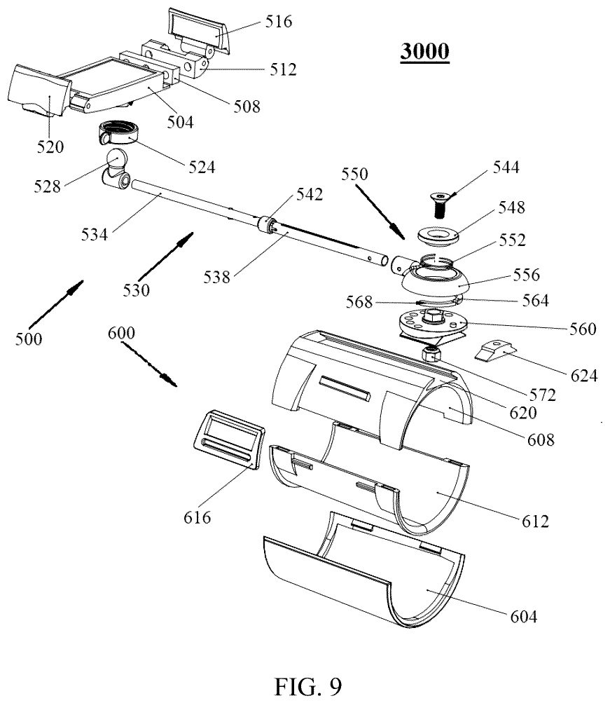

Referring to FIG. 9, there illustrated is an exploded view of a smart device support assembly 3000 according to a third exemplary embodiment of the present invention. The smart device assembly 3000 is designed for smartphone use, comprising a smart device holder 500 and a wrist brace 600.

In FIG. 9, the smart device holder 500 includes a phone mount 504, a clamp block 508, clamp hinge 512, two phone clamps, including clamps 516 and 520, collar lock 524, phone mount ball 528, and an appendage 530. The appendage 530 includes an inner appendage 534, an outer appendage 538, an arm cap 542 for locking the inner appendage 534 to the outer appendage 538, and a knuckle 550. The knuckle 550 includes a knuckle screw 544, a knuckle top 548, a knuckle spring 552, a knuckle top 556, a knuckle bottom 560, two knuckle stops 564 and 568, and a knuckle nut 572. The knuckle 550 of the appendage 530 is slidable in the glide track 620. The appendage 530 is retractable and expandable by either adjusting relative position of the inner appendage 534 to that of the outer appendage 538 before tightening the arm cap 542 to lock their relative positions or by sliding the appendage 530 in the glide track 620.

Referring again to FIG. 9, the wrist brace 600 comprises an outer cover 604, two semi-circular cuffs, including an upper cuff 608 and a lower cuff 612, a clasp 616, and a glide track 620. The knuckle nut 572 is fit into the glide track 620 to enable the appendage 530 slidable within the glide track 620. The clasp 616 tightens the upper cuff 608 with the lower cuff 612. An adjustment stop 624 is fit into one end of the glide track 620 to stop the appendage 530 from sliding off the track.

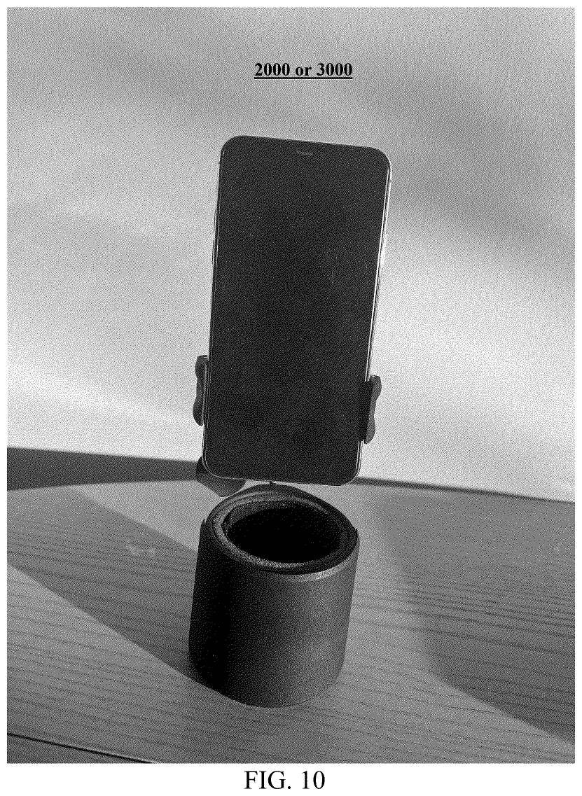

Referring to FIG. 10, there illustrated is an isometric view of a smartphone supported by the smart device support assembly 2000 or 3000.

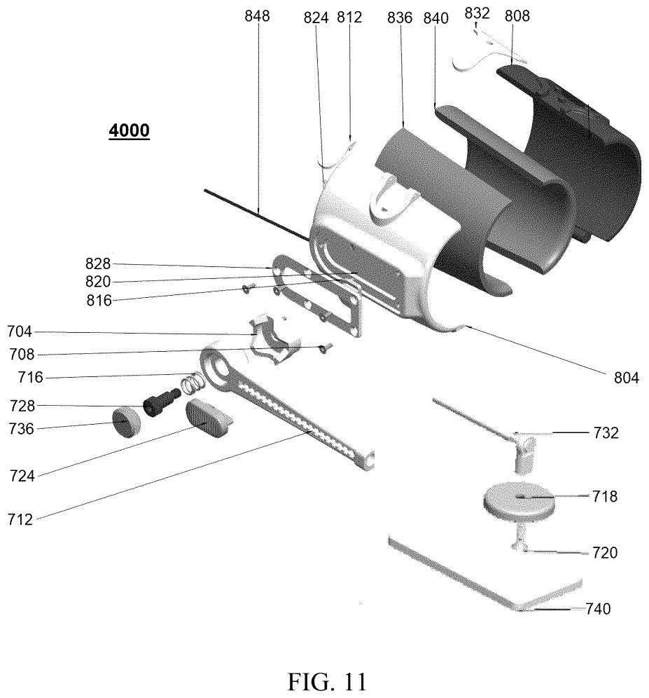

Referring to FIG. 11, there illustrated is an exploded view of a smart device support assembly 4000 according to a fourth embodiment of the present invention, showing various components of the smart device support assembly. The smart device assembly 4000 is designed for smartphone use, comprising a smart device holder 700 and a wrist brace 800.

In FIG. 11, the smart device holder 700 includes an appendage and a neodymium magnet 718 attached to one end of the appendage by a flat head screw 720. The magnet 718 is in a shape of round disk for binding the smartphone to the smart device holder. The appendage includes an outer appendage 712, a pivoting clutch female 704, an inner appendage 732, a compression spring 716, an alloy steel shoulder screw 728, a screw head mount knob 736, and a second slider 724.

The compression spring 716, the alloy steel shoulder screw 728, and the screw head mount knob 736 connect the outer appendage 712 to the pivoting clutch female 704. The pivoting clutch female 704 allows an upward vertical movement to 45-degree angle and back to 0 degree through manual manipulation. The appendage is moved with the pivoting clutch female 704. The second slider 724 fits into the outer appendage 712. One end of the inner appendage is connected to the magnet 718 and the other end of the inner appendage 732 is inserted into the hollow center section of the outer appendage 712 to be attached to the second slider 724. As a result, the appendage is expandable and retractable by sliding the inner appendage 732 along the outer appendage 712 using the second slider 724.

Referring again to FIG. 11, the wrist brace 800 comprises an outer cover including a top cover 808 and a bottom cover 804, an inner cuff including a top inner cuff and a bottom inner cuff, a clasp including a ratchet 832 and a ratchet cover 812, a first slider 816, and a slider cover 828. The ratchet 832 and the ratchet cover 812 are applied to tighten the top cover 808 and the bottom cover 804. The first slider 816, which is attached to the second slider 724, is slidable in the sliding track 820. The slider cover 828 covers the first slider 816 and a part of the sliding track 820. An extension spring with hook ends 824 assists in tightening the outer cover to the wrist of user. Hex drive flat head screws 708 tighten the slider cover 828 to the bottom cover 804

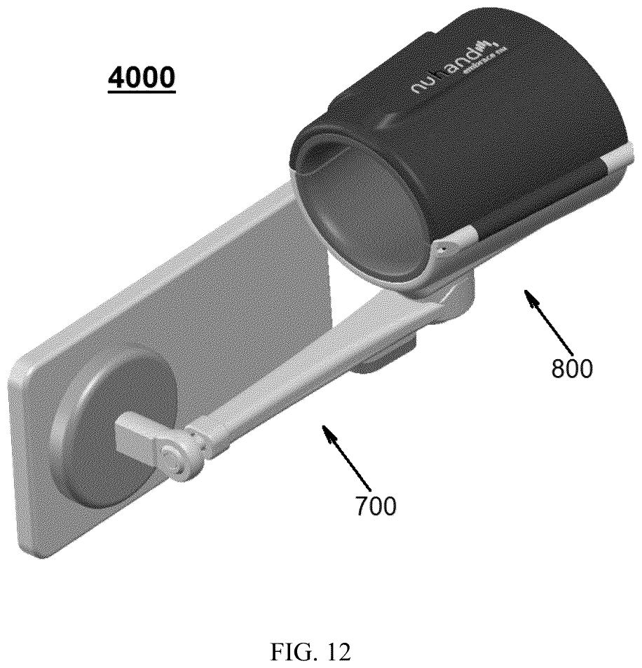

Referring to FIG. 12, there illustrated is an isometric back view of the smart device support assembly holding a smartphone according to the fourth embodiment 4000 of the present invention. The smart device support assembly comprises a device holder 700 and a wrist brace 800. As shown in FIG. 12, the smartphone 740 is bound to the smart device support assembly by the magnet 718. The magnet 718 is attached to one end of the appendage. The other end of the appendage in a shape of knuckle is connected to the pivoting clutch female 704 by the screw head mount knob 736, the shoulder screw 728, and the compression spring 716.

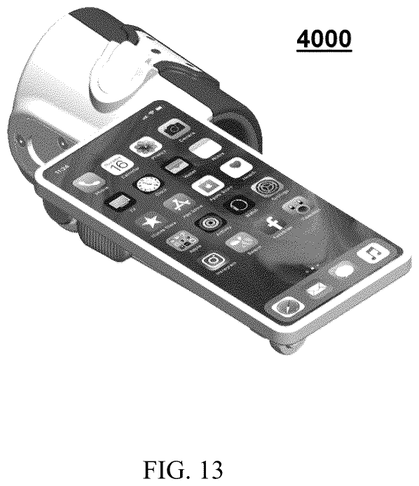

Referring to FIG. 13, there illustrated is an isometric front view of the smart device support assembly holding a smartphone according to the fourth embodiment 4000 of the present invention. As shown in FIG. 13, the clasp which includes the ratchet 832 and the ratchet cover 812 tightens the outer bottom cover 804 with the outer top cover 808.

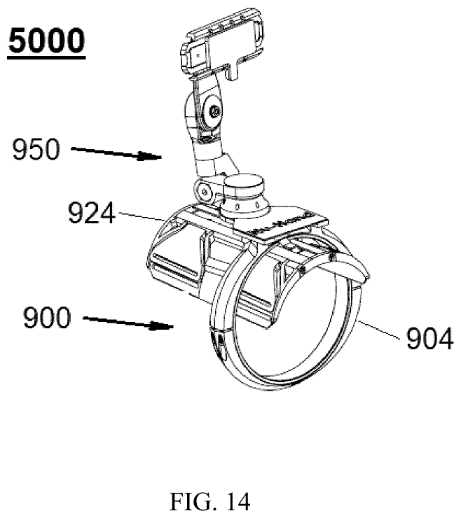

Referring to FIG. 14, there illustrated is an isometric view of the smart device support assembly according to the fifth embodiment of the present invention. The smart device assembly 5000 comprises a smart device holder 950 and a wrist brace 900.

In FIG. 14, the smart device holder 950 includes a device mount. Further, the device holder 950 comprises an arm base, an arm inner elbow, an arm outer elbow, an arm proximal, a female phone slide, a male phone slide, an arm plate, strap double slit, a ball socket mount, a ball joint O-ring, strap magnet, a strap termination, joint cap, and a sliding ring base.

Referring again to FIG. 14, the wrist brace 900 comprises a ring slider assembly 904 and an upper cuff 924 attached to an upper section of the interior circumference of the ring slider assembly 904.

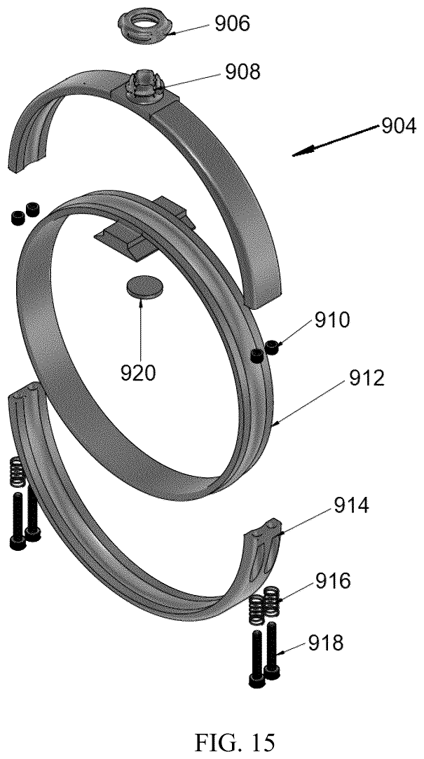

Referring to FIG. 15, there illustrated is an exploded view of the ring slider assembly 904 used in the smart device support assembly 5000 according to the fifth embodiment of the present invention. The ring slider assembly 904 includes a wrist mount 906, a ring slider top 908, a ring slider bottom 914, a ring slider base 912, and a magnet 920. Both ends of ring slider top 908 are embedded female threaded insert 910. The ring slider top 908 and the ring slider bottom 914 are tightened together onto the ring slider base 912 by socket head screws 918 screwed into the female threaded insert 910.

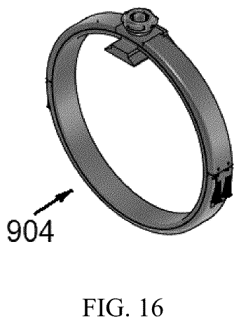

Referring to FIG. 16, there illustrated is an isometric view of an assembled ring slider assembly applied in the smart device support assembly according to the fifth embodiment of the present invention.

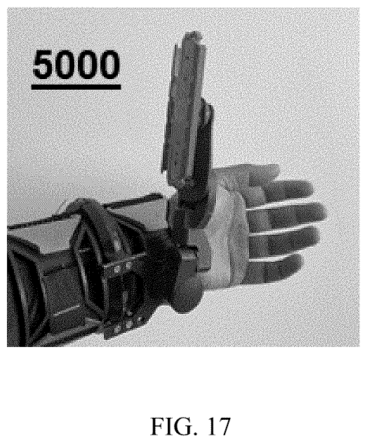

Referring to FIG. 17, there illustrated is an isometric view of the smart device support assembly held by a user on the front side of the hand according to the fifth embodiment of the present invention. As shown in FIG. 17, the user wears an inner cuff underneath of the wrist brace.

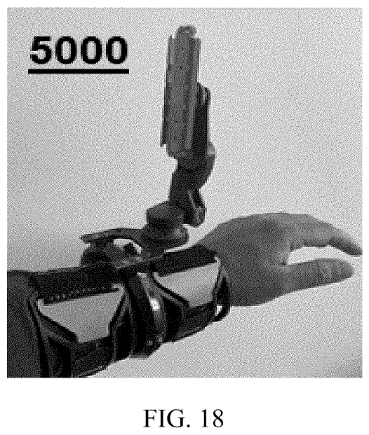

Referring to FIG. 18, there illustrated is an isometric view of the smart device support assembly held by a user on the back side of the hand according to the fifth embodiment of the present invention.

Various embodiments of the smart device support assemblies 1000, 2000, 3000, 4000, and 5000 of the present invention have been described. However, additional accessory pieces may be added to change the aesthetic and function of the primary device holder. For instance, a separate ring like structure piece can be attached to the device holder to allow for a smartphone to be positioned and used in different degrees of freedom around and above the wrist. For another instance, the wrist mount can have a recess designed to embed an electronic watch. Yet for another instance, there can be different wrist mount closing mechanisms, including clasp mechanism, sliding clasps, and sliding ratchet mechanism, in additional to the ones described in embodiments hereinabove.

A method of making various embodiments according to the present invention may include the following steps. The parts of the device holder and wrist brace, such as appendage, device mount, wrist mount, and semi-circular cuffs, may be manufactured from injection molding and then assembled with screws and bolts in such a way that the pieces work together, as described.

The wrist mount and the appendage could be made from plastics, thermoplastics, or aluminum. Or the wrist mount and attached appendage can be manufactured using different materials, such as that the wrist mount could be made from cloth fabric that can be either colorful or with different design patterns while the appendage could be made from plastics or aluminum.

An alternative way of making the smart device support assemblies 1000, 2000, 3000, 4000, and 5000 includes utilizing 3D printed materials and CNC machined metal aluminum parts. As the manufacturing process evolves, the material can range from customizable plastic for the wrist mounts, and various other materials for the device holder, such as other 3D printed material or other metals and plastics. The design of both the wrist mount and the device holder can be designed in various ways depending on the needs of the user and the ability of the engineer or product designer to create.

A method of using the smart device support assembly 1000-5000 of the exemplary embodiments of the present invention may include following steps: A person slides it on their wrist, and then connects their smart device to the appendage and manipulates the appendage as needed.

The present invention has been described in terms of exemplary embodiments solely for the purpose of illustration. Persons skilled in the art will recognize from this description that the invention is not limited to the embodiments described but may be practiced with modifications and alterations limited only by the spirit and scope of the appended claims. For example, future generations of the smart device support assembly 1000, 2000, 3000 and 4000 can have embedded sensors within the wrist mount to track various bodily functions or interact with the smartphone through Bluetooth and other Internet of Things (IoT) configurations.

What is claimed is:

1. A smart device support assembly comprising a wrist brace and a smart device holder supported by the wrist brace,
wherein the wrist brace includes two semi-circular cuffs and two sliding hinges attached to the cuffs, the sliding hinges allowing the two semi-circular cuffs adjustable in relative positions to fit over the wrist and forearm;
wherein the smart device holder includes a wrist mount, an expandable and retractable appendage, and a device mount, the appendage having one end attached to the wrist mount and the other end attached to the device mount;
wherein the wrist mount includes a glide track to guide sliding movement of the appendage; and
wherein the appendage includes an outer appendage, an inner appendage, and a slider, the inner appendage slidable in the outer appendage to make the appendage expandable and retractable, a bottom section of the slider fit into the glide track and an upper section of the slider connected to the outer appendage so that the outer appendage is slidable along the glide track.

2. The smart device support assembly of claim 1, wherein the smart device holder further includes a means of mounting the smart device on the device mount, the means selected from a group including a double-sided scorch tape.

3. The smart device support assembly of claim 1, wherein the appendage further comprises a first humeral head, a second humeral head, and tightening nuts, the first humeral head attached to a lower surface of one end of the outer appendage to face downward and the second humeral head attached to an upper surface of one end of the inner appendage to face upward; wherein the slider is connected to the outer appendage by being attached to the first humeral head through one of the tightening nuts; and wherein the device mount is connected to the inner appendage by being attached to the second humeral head through another one of the tightening nuts.

4. The smart device support assembly of claim 1, wherein the appendage further comprises a first snap fit and a second snap fit, the first snap fit is fit to the slider to guide and to prevent the slider from sliding off the glide track, and the second snap fit is fit to one end of the inner appendage to guide and to prevent the inner appendage from sliding off the outer appendage.

5. A smart device support assembly comprising a wrist brace and a smart device holder supported by the wrist brace,
wherein the wrist brace includes two semi-circular cuffs, a first clasp for tightening the two semi-circular cuffs together, and a glide track located along length of the wrist brace; and
wherein the smart device holder includes an appendage and a device mount, one end of the appendage having a knuckle fit in the glide track and the other end of the appendage attached to the device mount, the knuckle slidable in the glide track to allow the appendage moving along the longitude orientation of the wrist.

6. The smart device support assembly of claim 5, wherein the knuckle of the appendage is rotatable in a plane parallel to the glide track to allow different viewpoints on the screen of the smart device within easy access.

7. The smart device support assembly of claim 5, wherein the other end of the appendage is attached to the device mount through a ball joint that allows rotation of the smart device.

8. The smart device support assembly of claim 5, wherein the first clasp comprises a first set of magnets placed underneath the semi-circular cuffs for tightening the two cuffs together.

9. The smart device support assembly of claim 5, wherein the wrist brace further comprises two inner cuffs and a second clasp attached to one of the inner cuffs, the inner cuffs functioning as liners to the semi-circular cuffs and the clasp adjustable to size of a user's forearm.

10. The smart device support assembly of claim 5, wherein the underneath of the glide track is lined with a second set of magnets to allow the appendage sliding back and forth.

11. The smart device support assembly of claim 5, wherein the appendage further includes an outer appendage and an inner appendage fit into the outer appendage; and wherein the knuckle is attached to one end of the outer appendage, the appendage being retractable and expandable by adjusting relative position of the inner appendage to that of the outer appendage.

12. The smart device support assembly of claim 5, wherein the knuckle further includes a knuckle screw, a first knuckle top, a knuckle spring, a second knuckle top, a knuckle bottom, two knuckle stops, and a knuckle nut, the knuckle nut fit into the glide track to enable the appendage slidable in the glide track.

13. The smart device support assembly of claim 5 further comprising an adjustment stop fitting into one end of the glide track to stop the appendage from sliding off the track.

14. The smart device support assembly of claim 5, wherein the smart device holder further includes a clamp block, clamp hinge, two phone clamps, collar lock, and a phone mount ball.

15. A smart device support assembly comprising a wrist brace and a smart device holder supported by the wrist brace,
wherein the wrist brace includes a ring slider assembly and an upper cuff attached to an upper section of the interior circumference of the ring slider assembly, the ring slider assembly including a ring slider top, a ring slider base, and a ring slider bottom; and
wherein the smart device holder includes a device mount, an arm base, an arm inner elbow, an arm outer elbow, an arm proximal, a female phone slide, a male phone slide, and an arm plate.

16. The smart device support assembly of claim 15, wherein the wrist brace further includes female threaded inserts located within either end of the ring slider top, compression springs, and socket head screws passing through the compression springs to be screwed into the threaded inserts.

17. The smart device support assembly of claim 15, wherein the device holder further includes a strap double slit, a ball socket mount, a ball joint O-ring, strap magnet, a strap termination, joint cap, and a sliding ring base.

18. The smart device support assembly of claim 15, wherein the wrist brace further comprises an inner cuff fit inside of the ring slider assembly and under the upper cuff.

19. The smart device support assembly of claim 1, wherein the composition of the device mount comprises a plastic material selected from a group including polycarbonate.

20. The smart device support assembly of claim 1, wherein the composition of the device holder comprises a plastic material selected from a group including polycarbonate.
